(19) United States
(12) Patent Application Publication
(10) Pub. No.: US 2011/0034413 A1
(43) Pub. Date: Feb. 10, 2011

(54) NOVEL QUERCETIN DERIVATIVES AS ANTI-CANCER AGENTS

(76) Inventors: Narendra Shriram Joshi, Uttar Pradesh (IN); Pawan Aggarwal, Uttar Pradesh (IN); Vitthalbhai Ketan Hirpara, Uttar Pradesh (IN); Manu Jaggi, Uttar Pradesh (IN); Anu Singh, Uttar Pradesh (IN); Anshumali Awasthi, Uttar Pradesh (IN); Ritu Verma, Uttar Pradesh (IN)

Correspondence Address: LADAS & PARRY LLP, 26 WEST 61ST STREET, NEW YORK, NY, 10023, US

(21) Appl. No.: 12/676,709

(22) PCT Filed: Aug. 8, 2008
(86) PCT No.: PCT/IN2008/000496

§ 371 Date: Oct. 25, 2010

(30) Foreign Application Priority Data
Aug. 9, 2007 (IN) 1693/DEL/2007

(51) Int. Cl. A61K 31/7008; C07D 311/30; C07H 5/06; A61K 31/352; A61P 35/00

(52) U.S. Cl. 514/62; 549/403; 536/55.2; 536/18.7; 514/456; 514/23

(57) ABSTRACT

The present invention provides novel Quercetin derivatives of formula (I) and pharmaceutically acceptable salts, hydrates, and solvates thereof,

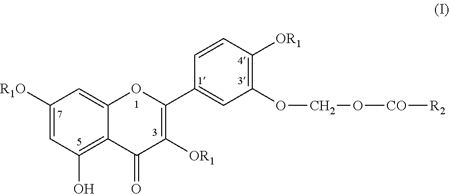

wherein R₁ is hydrogen, benzyl or substituted benzyl; R₂ is hydrogen, benzyl or substituted benzyl, linear or branched (C₁-C₆) alkyl, substituted alkyl, aryl, substituted aryl, heterocycle and substituted heterocycle, useful for treatment of various disorders including cancer, multi-drug resistant cancers, viral infections etc. The invention also provides a process for the preparation of compounds of formula (I) and pharmaceutical compositions comprising the same.

### FIELD OF THE INVENTION

The present invention relates to novel Quercetin derivatives of formula (I) and pharmaceutically acceptable salts, hydrates, and solvates thereof; a process for the preparation of the novel Quercetin derivatives of formula (I), and pharmaceutically acceptable salts, hydrates, and solvates thereof; and pharmaceutical compositions comprising the same.

The present invention also relates to novel Quercetin derivatives of formula (I) and pharmaceutically acceptable salts, hydrates, solvates thereof that are useful for the treatment of various disorders including cancer, multi-drug resistant cancers, viral infections etc.

### BACKGROUND OF THE INVENTION

The Chemical entity, 2-(3,4-Dihydroxyphenyl)-3,5,7-trihydroxy-4H-1-benzopyran-4-one, generically known as Quercetin of formula (II),

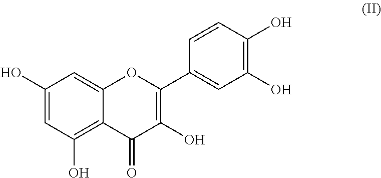

is a naturally occurring flavonoid isolated from the plant, Rhododendron cinnabarinum Hook of the family Erricaceae, first reported by Rangaswami et al., in “Proc. Indian Acad. Sci.” 1962, 56 A, 239.

Quercetin is the aglycon of quercitrin, rutin, and of other glycosides. It is widely distributed in the plant kingdom, especially in rinds and barks, in clover blossoms and in ragweed pollen. Being a polyphenolic natural organic compound, it is one of the large numbers of water-soluble plant pigments called flavonoids (meaning class of plant secondary metabolites known for their antioxidant activity) that are largely responsible for the color of many flowers, fruits and vegetables. Higher concentrations of Quercetin are available in apples, onions, tea and red wine. Other sources of Quercetin include olive oil, grapes, broccoli, cauliflower, cabbage, dark cherries and dark berries such as blueberries, blackberries and bilberries.

Quercetin is a powerful natural antioxidant and a dietary flavonoid and is found to be the most effective inhibitor of oxidative damage to LDL (bad) cholesterol in vitro, thereby reducing the risk of developing atherosclerosis. The average U.S. citizen eating a normal, healthy diet including fruits and vegetables consumes approximately 25-50 mg of Quercetin per day. Quercetin and other flavonoids (also referred to as bioflavonoids) cannot be produced in the human body.

Owing to its significant antioxidant capacity as well as several other biological activities, such as coronary vasorelaxation (meaning reduction in tension of the blood vessel walls) properties, anti-diabetic, natural antihistamine, anti-inflammatory, unique ability to inhibit TNF-alpha (a cytokine involved in systemic inflammation) gene expression, antiviral, enhancement of the immune system and helping in maintaining mental performance, and others.

Studies have shown that Quercetin exhibits anticancer effects. A number of phase I clinical trials have been performed with Quercetin evaluating pharmacokinetics (Clin Cancer Res., 1996 April; 2(4), 659-68 and adenoma regression (Clin. Gastroenterol. Hepato., 2006 Aug 4(8):1035-38. A combination of Curcumin and Quercetin was evaluated to regress the adenomas in patients with familialadenomatous polyposis (FAP), an autosomal-dominant disorder characterized by the development of colorectal adenomas and eventual colorectal cancer. The study found that the combination appeared to decrease polyp numbers and size from baseline after 6 months of treatment. Owing to the aforementioned properties the Quercetin skeleton is emerging as a new prototype for treatment of cancer.

However, the clinical developments of Quercetin have been hampered owing to its extreme water insolubility and a limited solubility in pharmaceutically acceptable solvents.

In view of the above, there have been efforts to produce analogs or derivatives of Quercetin having improved aqueous solubility and which, moreover, would be more suitable for use as a Pharmaceutical.

Several chemical modifications of Quercetin (II) have been carried out at varied positions of the skeleton from the perspective of synthesis and production of:

i) Novel Quercetin derivatives with improved aqueous solubility; and
ii) Novel Prodrugs for improved release of Quercetin in blood plasma,
with the objective of finding out a clinically useful anti-cancer or anti-proliferative agent. (Chemische Berischte., 1975, 108 (5):1482-501; Anticancer Research, 2000, 20 (1A): 271-277; Annals of Oncology, 2001, 12, 245-248; J. of Med. Chem., 2005, 48 (8), 2790-2804; and Letters in Organic Chemistry, 2005, 2 (6), 535-538). As a result, QC-12 of formula (IV),

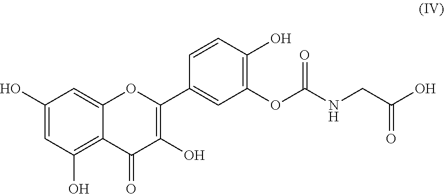

has emerged as a potential anticancer agent, which is in Phase I clinical trial.

It might also be mentioned that variations of substituents at the positions 3, 5, 7, 3′ and 4′ of Quercetin of formula (II), has been the subject matter of research efforts to obtain potential lead compounds, viz. U.S. Pat. No. 3,420,815; U.S. Pat. No. 4,202,815;U.S. Pat. No. 6,235,294; JP 07010898; U.S. Pat. No. 5,565,435; U.S. Pat. No. 5,955,100; and U.S. Pat. No. 6,258,840.

Even though, all the above mentioned reports collectively disclose a large number of Quercetin derivatives, with a vast majority of them found to possess anti-cancer as well as other biological activities, however, due to various reasons they are not particularly good candidates, clinically as well as do not have the best of pharmacokinetic properties.

There exists a need, therefore, for further structural modifications in Quercetin skeleton to establish a meaningful structure activity relationship as well as to obtain more potent anticancer and antiviral agents.

In their endeavors to find novel Quercetin derivatives useful as anticancer and antiviral agents, which are not only potent, therapeutically but also clinically acceptable, the present inventors have synthesized a number of Quercetin derivatives of formula (I) and evaluated them for various disorders including their cytotoxic profile directly using cancer, multi drug resistant (MDR) cancer and normal cell lines.

### OBJECT OF THE PRESENT INVENTION

An object of the present invention is to provide novel Quercetin derivatives for treatment of various disorders including cancer and multi-drug resistant cancers.

Another object of the present invention is to provide novel Quercetin derivatives for treatment of various viral infections.

Yet another object of the invention is to provide processes for preparation of novel Quercetin derivatives.

A further objective of the present invention is to provide pharmaceutical compositions comprising novel Quercetin derivatives for the treatment of various disorders including cancers, multi-drug resistant cancers and viral infections.

Still further object of the present invention is to provide a method of treatment of various disorders including cancers, multi-drug resistant cancers and viral infections through administration of novel Quercetin derivatives.

### SUMMARY OF THE INVENTION

In one aspect, the present invention provides novel 3,5,7,3′,4′ substituted Flavonoid derivatives also known as Quercetin derivatives of formula (I) and pharmaceutically acceptable salts, hydrates, and solvates thereof,

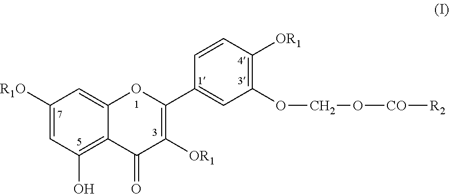

wherein R₁ is hydrogen, benzyl or substituted benzyl; R₂ is hydrogen, benzyl or substituted benzyl, linear or branched (C₁-C₆) alkyl, substituted alkyl, aryl, substituted aryl, heterocycle and substituted heterocycle.

In another aspect, the present invention provides a process for preparation of the novel Quercetin derivatives of formula (I), and pharmaceutically acceptable salts, hydrates, and solvates thereof comprising the steps of:
i) reaction of a flavanoid compound of formula (III),

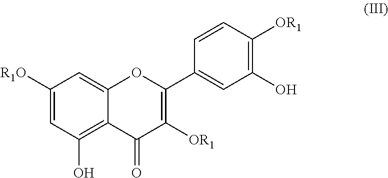

wherein R₁ is benzyl or substituted benzyl with a halo alkyl ester of Formula (V),

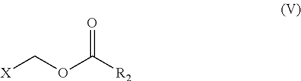

wherein X is chloro, bromo, or iodo and R₂ is hydrogen, benzyl or substituted benzyl, linear or branched (C₁-C₆) alkyl, substituted alkyl, aryl, substituted aryl, heterocycle and substituted heterocycle in presence of a base and in presence of a suitable solvent to give compound of formula (I), wherein R₁ is benzyl or substituted benzyl; and
ii) subjecting the compound of formula (I), as obtained in step (i), wherein R₁ is benzyl or substituted benzyl to catalytic hydrogenation to obtain compound of formula (I), wherein R₁ is hydrogen.

The pharmaceutically acceptable salts of compound of formula (I), wherein R₁ is benzyl or substituted benzyl or hydrogen can be prepared from the corresponding compounds of formula (I), wherein R₁ is benzyl or substituted benzyl or hydrogen, as obtained in steps i) or ii) by method known in the art.

In yet another aspect, the present invention provides pharmaceutical compositions comprising a therapeutically effective amount of the novel Quercetin derivatives of formula (I) and pharmaceutically acceptable salts, hydrates and solvates thereof that are useful for the treatment of various disorders including cancer, multi-drug resistant cancer, and viral infections in humans.

In a further aspect, the present invention a method of treatment of various disorders including cancer, multi-drug resistant cancer, and viral infections in humans comprising administration of the novel Quercetin derivatives of formula (I) and pharmaceutically acceptable salts, hydrates and solvates thereof.

In a still further aspect, the present invention provides novel Quercetin derivatives of formula (I) and pharmaceutically acceptable salts, hydrates and solvates thereof, which are useful for inhibition of tumor cancer cells including multi drug resistant (MDR) cancer cells.

### DETAILED DESCRIPTION OF THE INVENTION

As mentioned hereinbefore, the present invention provides novel Quercetin derivatives of formula (I) and pharmaceutically acceptable salts, hydrates, and solvates thereof,

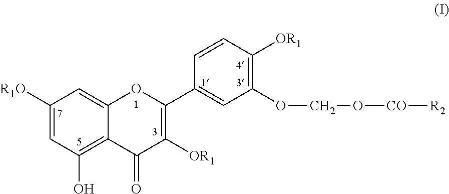

wherein R₁ is hydrogen, benzyl or substituted benzyl; R₂ is hydrogen, benzyl or substituted benzyl, linear or branched (C₁-C₆) alkyl, substituted alkyl, aryl, substituted aryl, heterocycle and substituted heterocycle.

Further, as mentioned hereinbefore, the present invention provides a process for preparation of the Novel Quercetin derivatives of formula (I), a process for preparation of the novel Quercetin derivatives of formula (I), and pharmaceutically acceptable salts, hydrates, and solvates thereof comprising the steps of:
i) reaction of a flavanoid compound of formula (III),

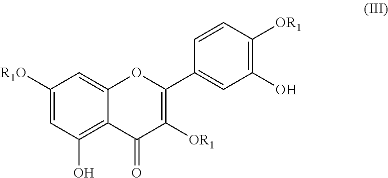

wherein R₁ is benzyl or substituted benzyl with a halo alkyl ester of Formula (V),

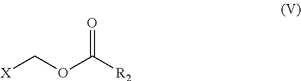

wherein X is chloro, bromo, or iodo and R₂ is hydrogen, benzyl or substituted benzyl, linear or branched (C₁-C₆) alkyl, substituted alkyl, aryl, substituted aryl, heterocycle and substituted heterocycle in presence of a base and in presence of a suitable solvent to give compound of formula (I), wherein R₁ is benzyl or substituted benzyl; and
ii) subjecting the compound of formula (I), as obtained in step (i), wherein R₁ is benzyl or substituted benzyl to catalytic hydrogenation to obtain compound of formula (I), wherein R₁ is hydrogen.

The flavanoid compound of formula (III), wherein R₁ is benzyl or substituted benzyl may be obtained as per the methods reported by Mohamed Bouktaib et al. in Tetrahedron, 2002, 58, 10001-10009.

In a typical embodiment, the flavanoid compound of formula (III), wherein R₁ is benzyl or substituted benzyl is reacted with a suitable halo alkyl ester of formula (V) in the presence of a base and in the presence of a suitable solvent at a temperature of between 10° C. to 80° C. to produce the corresponding compounds of formula (I), wherein R₁ is benzyl or substituted benzyl. The reaction is complete in about 4 to 8 hours and the compounds of formula (I), wherein R₁ is benzyl or substituted benzyl thus produced is isolated from the reaction mixture by suitable methods.

The halo alkyl esters of formula (V) are employed in stoichiometric proportions or in slight excess of the stoichiometric proportions of the of the flavanoid compound of formula (III). Typically, the halo alkyl esters of formula (V) are employed in proportions of between 1 to 1.5 moles per mole of the flavonoid compound of formula (III).

Suitable solvents that can be employed for the reaction of the flavanoid compound of formula (III) and the halo alkyl ester of formula (V) are preferably aprotic in nature and such aprotic solvents that can be employed include N,N-dimethylformamide, N,N-dimethylacetamide, dioxane, tetrahydrofuran, acetonitrile, acetone, dichloromethane, dichloroethane and the like, of which N,N-dimethylformamide is the preferred aprotic solvent.

Both organic and inorganic bases can be employed for reaction of the flavanoid compound of formula (III) and the halo alkyl ester of formula (V).

The organic bases that can be employed include tertiary amines like alky amines, pyridine, 2,6-lutidine, N-methyl-morpholine, 4-dimethylaminopyridine, N,N-dimethylaniline. Alky amines are preferred and amongst such alkyl amines, triethyl amine is preferred.

The inorganic bases that can be employed include alkali metal carbonates, such as sodium carbonate, potassium carbonate, lithium carbonate; and alkali metal bicarbonates, such as, sodium bicarbonate or potassium bicarbonate. Alkali metal carbonates are more preferred and amongst the alkali metal carbonates, potassium carbonate is preferred.

Generally the base is employed in stoichiometric proportions or in excess of the stoichiometric proportions of the of the flavanoid compound of formula (III). Typically, the base is employed in proportions of between 1 to 2.0 moles per mole of the flavonoid compound of formula (III).

Upon completion of the reaction the compound of formula (I), wherein R₁ is benzyl or substituted benzyl can be isolated by suitable methods. When water-miscible aprotic solvents like N,N-dimethylformamide, N,N-dimethylacetamide, dioxane, tetrahydrofuran, acetonitrile, acetone and the like are employed in the reaction, the compound of formula (I) can be isolated by dilution of the reaction mixture with water and collecting the precipitated compound of formula (I) or extracting the diluted reaction mixture with a water-immiscible organic solvent and isolation of the compound of formula (I) from the water-immiscible organic solvent by evaporation of the solvent or crystallization of the product from the same or precipitation of the product from the same by addition of a co-solvent. When water-immiscible aprotic solvents like dichloromethane, dichloroethane and the like are employed in the reaction, the compound of formula (I) can be isolated by dilution of the reaction mixture with water, followed by separation of the organic and aqueous phases and isolation of the compound of formula (I) from the water-immiscible organic solvent by evaporation of the solvent or crystallization of the product from the same or precipitation of the product from the same by addition of a co-solvent.

Compounds of formula (I) wherein R₁ is hydrogen are obtained by catalytic hydrogenation of the compounds of formula (I), wherein R₁ is benzyl or substituted benzyl.

In general, the catalytic hydrogenation is carried out by subjecting the compounds of formula (I), wherein R₁ is benzyl or substituted benzyl to hydrogen pressure in the presence of an organic solvent and a hydrogenation catalyst at a hydrogen pressure from about 0 to 200 psi and at a temperature of from about 10° C. to 40° C.

Typical hydrogenation catalysts that can be employed are selected from those of Palladium or Platinum supported on Carbon. Palladium supported on carbon is more preferred.

Suitable organic solvents that can be employed for the catalytic hydrogenation reaction are those selected from the class of organic acids, cycloethers, alcohols and mixtures thereof. Suitable organic acids that can be employed include acetic acid, butenic acid, propionic acid and the like; suitable cycloethers include tetrahydrofuran, dioxane and the like; suitable alcohols include methanol, ethanol, propanol and the like. Amongst the preferred organic solvents are those belonging to the class of cycloethers and alcohols and the preferred solvents are tetrahydrofuran and ethanol.

Upon completion of the reaction the compound of formula (I), wherein R₁ is hydrogen After removal of the hydrogenation catalyst from the reaction mixture, the compound of formula (I) can be isolated by dilution of the reaction mixture with water and collecting the precipitated compound of formula (I) or extracting the diluted reaction mixture with a water-immiscible organic solvent and isolation of the compound of formula (I) from the water-immiscible organic solvent by evaporation of the solvent or crystallization of the product from the same or precipitation of the product from the same by addition of a co-solvent.

Compounds of formula (I), wherein R₁ is benzyl or substituted benzyl as well as compounds of formula (I), wherein R₁ is hydrogen depending on the solvents utilized for their preparation, isolation, and crystallization may further be isolated as hydrates and solvates and such hydrates and solvates are construed to be within the scope and spirit of the present invention.

Representative pharmaceutically acceptable salts of the novel Quercetin derivatives of formula (I), wherein R₁ is benzyl or substituted benzyl as well as compounds of formula (I), wherein R₁ is hydrogen include but are not limited to those salts such as ascorbate, acetate, benzoate, citrate, fumarate, gluconate, glutamate, hydrochloride, hydrogen sulfate, lactate, oxalate, phosphate, diphosphate, stearate, succinate, sulfate, tartarate, trifluoroacetate and valerate; Al, Ca, Li, Mg, Na and K salts; halides; salts of amino acids such as ammonium, substituted ammonium, glycine, alanine, lysine, arginine, or guanidine salts; amino sugar salts such as N-methyl-D-glucamine (meglumine), 1-amino-1-deoxy-D-sorbitol, 1-deoxy-1-(methylamino-D-galactitol, 1-deoxy-1-(octylamino)-D-glucitol, 1-deoxy-1-(2-hydroxyethylamino)-D-glucitol, disorbytylamine, D-galactosamine, D-glucosamine, D-mannosamine and the like.

The abovementioned salts of the novel Quercetin derivatives of formula (I), wherein R₁ is benzyl or substituted benzyl as well as compounds of formula (I), wherein R₁ is hydrogen can be prepared by employing methods known in the art.

Representative compounds of formula (I) prepared as per the method of the present invention and referred to as Compound Nos. 1 to 15 are summarized in Table-I.

TABLE I
Representative Novel Quercetin Derivative Compounds of Formula (I)
Prepared as per the Method of the Present Invention

| Compound No. | R₁ | R₂ |
|---|---|---|
| 1 | Benzyl | —C(CH₃)₃ |
| 2 | Benzyl | —CH₃ |
| 3 | Benzyl | —C₃H₇ |
| 4 | Benzyl | —CH(CH₃)₂ |
| 5 | Benzyl | —C₄H₉ |
| 6 | Benzyl | —CH₂—CH(CH₃)₂ |
| 7 | H | —C(CH₃)₃ |
| 8 | H | —CH₃ |
| 9 | H | —C₃H₇ |
| 10 | H | —CH(CH₃)₂ |
| 11 | H | —C₄H₉ |
| 12 | H | —CH₂—CH(CH₃)₂ |
| 13 | Benzyl | —CH₂CH₂CH₃ |
| 14 | H | —CH₂CH₂CH₃ |
| 15 | Benzyl | —CH₂CH₃ |

The method for preparation of the novel Quercetin derivative Compounds Nos. 1 to 15 of the present invention is delineated in Scheme-I.

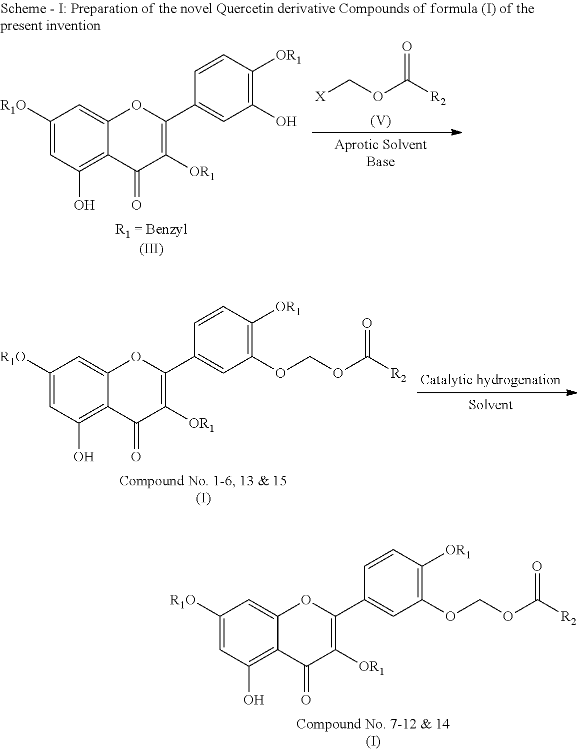

Further, aqueous solubility, plasma stability and anticancer potential of the novel Quercetin derivatives of formula (I) have also been studied and subsequently compared with QC-12 of formula (IV) and Quercetin of formula (II).

The aqueous solubility of the novel Quercetin derivatives of formula (I) were determined and subsequently compared with QC 12. 500 μM solutions of the various novel Quercetin derivatives were prepared in aqueous media from DMSO stock solution. The samples were scanned in UV spectrophotometer at its λₘₐₓ to obtain optical density (O.D) of the compound. Concentration against the OD value was obtained by calibration curve constructed by plotting the Optical density Vs concentration of calibration standards of the compounds.

Calibration standards were prepared by diluting the stock solution of the compound further in suitable solvents ensuring the overall compound solubility. Calibration standard of concentration 500 μM, 200 μM, 50 μM, 12.5 μM and 3.13 μM were prepared. These calibration standards were scanned in UV spectrophotometer at its highest wavelength (λₘₐₓ) to obtain the optical density values (OD). A standard curve was constructed by plotting the Optical density Vs concentration.

A comparison of the aqueous solubility of the some of the novel Quercetin derivatives of formula (I) with that of Quercetin (II) and QC-12 (IV) is summarized in Table-II, from which it can be seen that Compound No. 14 exhibits solubility comparable to QC-12 (IV), whereas Compound Nos. 7 and 8 exhibit better solubility than Quercetin (II).

TABLE II
Comparison of the Aqueous Solubility of the novel Quercetin
derivatives formula (I) and that of Quercetin (II) and QC-12 (IV)

| Compound No. | Solubility in μM |
|---|---|
| 13* | <3.13 |
| 14* | 199 |
| 15* | <3.13 |
| 1* | 1.48 |
| 7* | 30.568 |
| 8* | 16.525 |
| 2* | 0.591 |
| Quercetin (II) | 4.022 |
| QC-12 (IV) | >200 |

*Corresponding to the novel Quercetin derivatives of formula (I) of the present invention, as summarized in Table-I.

The Plasma stability of the novel Quercetin derivatives of formula (I) were evaluated by determining their half-life in plasma and subsequently compared with QC-12 (IV). Plasma samples of concentration 100 μM spiked with the compounds were incubated at 37° C. and at specified time point the samples were quenched using chilled acetonitrile. Samples were then analyzed using liquid chromatography to determine the concentration at respective time point. Half-life was calculated from the logarithmic curve drawn between time and concentration.

A comparison of the half life of some of the novel Quercetin derivatives of formula (I) with that of QC-12 (IV) is summarized in Table-III.

TABLE III
Comparison of the Half Life of the novel Quercetin
derivatives of formula (I) with that of QC-12 (IV)

| Compound No | Half life(in mins) |
|---|---|
| 14* | <3.0 |
| 7* | 6.24 |
| 8* | <3.0 |
| QC-12 (IV) | 43.87 |

*Corresponding to the novel Quercetin derivatives of formula (I) of the present invention, as summarized in Table-I.

The anticancer potential of the novel Quercetin derivatives of formula (I) were evaluated and subsequently compared with Quercetin (II). The anticancer potential was determined by the colorimetric MTT conversion assay of Mossman. Human cancer cells representing ovary (PA-1; SK-OV-3); prostate (DU 145); lung (A-549) and normal fibroblast (NIH-3T3) were separately seeded at density of 1000 cells/well into 96 well plates in 180 μl of culture medium with 10% fetal calf serum. After 24 h, cells were incubated with different concentrations of the novel Quercetin derivatives ranging from 10 μM to 100 μM with relevant controls at 37° C. in a CO₂ incubator in triplicate wells. The exposure medium (Quercetin derivatives in culture medium) of all the cells was refreshed after every 24 h. Cells were incubated with the derivatives for total of 72 h. The assay was terminated by the addition of 20 μl, of MTT solution (5 mg/ml) in each well and percentage cytotoxicity was calculated as given below. The IC₅₀ values were determined by nonlinear regression using Prism software v 4.01.

Percentage Cytotoxicity=100×[1−(X/R₁)], where X=absorbance of treated sample at 540 nm

A comparison of the cytotoxic potential of some of the novel Quercetin derivatives of formula (I) with that of Quercetin (II) is summarized in Table-IV, from which it can be seen that Compound Nos. Compound No. 7, 8, and 14 have demonstrated better cytotoxic potential than Quercetin (II).

TABLE IV
Comparison of the Cytotoxic Potential of the novel
Quercetin derivatives of formula (I) with Quercetin
(II) using different Cancer Cell Lines

| | | IC₅₀ Value (μM) | | | | |
|---|---|---|---|---|---|---|
| Sr. | Compound | SKOV3 | PA-1 | DU 145 | A 549 | NIH 3T3 |
| No. | No. | (Ovary) | (Ovary) | (Prostate) | (Lung) | (Normal) |
| 1 | 1* | >100 | >100 | >100 | — | >100 |
| 2 | 7* | >100 | 20.88 | 83.11 | — | >100 |
| 3 | 2* | >100 | >100 | >100 | — | >100 |
| 4 | 8* | >100 | 19.35 | >100 | — | >100 |
| 5 | 13* | >100 | >100 | >100 | >100 | >100 |
| 6 | 14* | 94.63 | <10 | >100 | 61.86 | >100 |
| 7 | 15* | >100 | 10.7 | >100 | >100 | >100 |
| 8 | Quercetin | 88.39 | 35.33 | >100 | 83.74 | 68.67 |
| | (II) | | | | | |

*Corresponding to the novel Quercetin derivatives of formula (I) of the present invention, as summarized in Table - I.

Pharmaceutical compositions comprising the novel Quercetin derivatives of formula (I) are made up or formulated for administration in any suitable manner in the course of medical treatment, for example parentally, including intravenously, intramuscularly and subcutaneously or orally. Such pharmaceutical compositions containing or incorporating, conveniently in unit dosage form, a therapeutically effective amount of the novel Quercetin derivatives of formula (I), or the equivalent of a therapeutically effective amount of the novel Quercetin derivatives of formula (I), together possibly with at least one other ingredient providing a compatible pharmaceutically acceptable additive, carrier, diluent or excipient, may be prepared by any of the methods 1 known in the art of pharmacy.

Typical carriers that can be employed include lubricants and diluents. Suitable diluents may include RPMI 1649, buffered saline, isotonic NaCl, Ringer's solution, water, distilled water, polyethylene glycol, 2% Tween in water, 50% dimethylsulfoxide in water (v/v), propylene glycol, phosphate buffered saline, balanced salt solution, glycerol, and other conventional fluids that are suitable for intravenous administration.

In addition, the composition can contain other additives, such as suspending agents, thickening agents, sweeteners, preservatives, bulking agents and flavouring agents.

The sweeteners that can be used include sugars such as fructose, sucrose, glucose, maltose, or lactose as well as non caloric sweetener such as aspartame, which can be used alone or in combination with another non-caloric or low caloric sweetener known to have synergistic sweetening properties with aspartame, e.g. saccharin, acesulfame, thaumatin, chalcone, cyclamate, stevioside and the like.

The water soluble preservatives found useful in the present invention include sodium benzoate, sodium citrate and benzalkonium chloride, the preferred one being sodium benzoate and the like.

Suitable bulking agents are lactose, mannitol, isomalts, polydextrose, starch, microcrystalline cellulose, sorbitol, calcium sulphate, calcium phosphate, acacia and the like. Representative flavouring liquids include, artificial, natural or synthetic fruit flavours such as lemon, orange, banana, grape, lime, apricot and grapefruit oils and fruit essences including apple, strawberry, cherry, orange, pineapple and so forth; bean and nut derived flavours such as coffee, cocoa, cola, peanut, almond and so forth; and root derive flavours such as licorice.

The synthesis of the novel Quercetin derivatives of formula (I) is further described in the following examples, which however should not be constructed as limiting scope of the invention.

Quercetin (II) was purchased from Ws Shanghai Worldbest Industry Development Imp. & Exp. Co. Ltd., China.

3,7-bis(benzyloxy)-2-(4-(benzyloxy)-3-hydroxyphenyl)-5-hydroxy-4H-chromen-4-one (also known as tribenzyloxy Quercetin), of formula (III) was prepared from Quercetin as per the method disclosed in Tetrahedron, 2002, 58, 10001-10009.

The following abbreviations are used in the Examples that follow: DCM (Dichloromethane), DMSO (Dimethyl Sulphoxide), THF (Tetrahydrofuran) and Pd (Palladium).

### Example-1

### Procedure for the Synthesis of Compound as Described Under Formula (III, Wherein R₁=benzyl): 3,7-Bis-benzyloxy-2-(4-benzyloxy-3-hydroxy-phenyl)-5-hydroxy-chromen-4-one

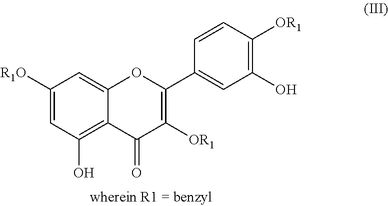

Benzyl bromide (174.5 g, 1.02 mol) was added drop wise to a solution of Quercetin (II, 100 g, 0.3 mol) in DMF (1.4 lt), potassium carbonate (165.6 gm, 1.2 mol) at 60° C. under nitrogen. Reaction mixture stirred for 3 h at 60-62° C. The reaction mixture was diluted with ethyl acetate (3.5 lt) and water (2 lt). The organic layer was separated, washed with water, dried over anhydrous sodium sulphate and evaporated to give crude product. The crude product was purified by column chromatography (60-120 mesh silica gel) using Methylene chloride/Hexane as eluent to furnish the required product.

### Yield 65 g (38.4%).

R_{f} 0.67 (30% Ethyl acetate/Petroleum ether);

¹HNMR (DMSO-d₆): δ 5.0 (s, 2H), 5.20 (s, 2H), 5.22 (s, 2H), 6.45-6.46 (d, 1H), 6.79-6.80 (d, 1H), 7.10-7.13 (d, 1H), 7.27-7.54 (m, 18H), 9.4 (s, 1H); MS (ES+) m/z 573.3 (M+H).

### Example-2

### Procedure for the Synthesis of Compounds as Described Under Formula (I)

### Compound No. 1: (2-(Benzyloxy)-5-(3,7-bis(benzyloxy)-5-hydroxy-4-oxo-4H-chromen-2-yl)phenoxy)methyl pivalate

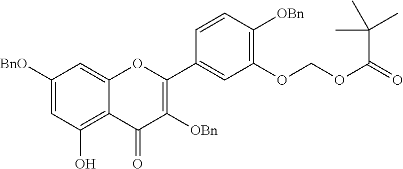

Chloromethylpivalate (0.55 ml, 3.8 mmol) was added drop wise to a suspension of 3,7-bis(benzyloxy)-2-(4-(benzyloxy)-3-hydroxyphenyl)-5-hydroxy-4H-chromen-4-one [(III, R₁=benzyl) 2 g, 3.4 mmol], Potassium carbonate (0.72 g, 5.2 mmol) in N,N-dimethylformamide (20 ml) at 0° C. The resulting mixture was stirred at 0° C. for ten minutes and further for 8 hour at 25-28° C. The reaction mixture was diluted with ethyl acetate (50 ml) and water (50 ml). The organic layer was separated, washed with water, dried over anhydrous sodium sulphate and evaporated to give crude product. The crude product was purified by column chromatography (60-120 mesh silica gel) using methylene chloride/Hexane as eluent to furnish the required product. Yield 1.9 g (79.1%).

R_{f} 0.73 (30% Ethyl Acetate/Petroleum ether); ¹H NMR (DMSO-d₆): δ 1.18 (s, 9H), 5.06 (s, 2H), 5.19 (s, 2H), 5.23 (s, 2H), 6.47-6.48 (d, 1H), 6.90-6.91 (d, 1H), 7.29-7.47 (m, 19H), 7.79-7.80 (d, 1H), 7.93-7.97 (dd, 1H).

### Compound No. 2: (2-(Benzyloxy)-5-(3,7-bis(benzyloxy)-5-hydroxy-4-oxo-4H-chromen-2-yl)phenoxy)methyl acetate

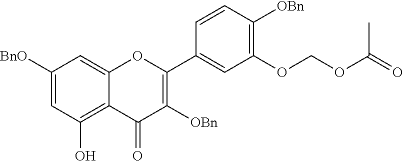

Bromomethylacetate (0.2 ml, 2.0 mmol) was added drop wise to a suspension of 3,7-bis(benzyloxy)-2-(4-(benzyloxy)-3-hydroxyphenyl)-5-hydroxy-4H-chromen-4-one [(III, R₁=benzyl) 1 g, 1.7 mmol], triethylamine (0.48 ml, 3.4 mmol) in methylene chloride (25 ml), at 25° C. The resulting mixture was stirred at 25° C. for ten minutes and further for 4 hour at 40-42° C. The reaction mixture was diluted with methylene chloride (25 ml) and water (50 ml). The organic layer was separated, washed with water, dried over anhydrous Sodium sulphate and evaporated to give crude product. The crude product was purified by crystallization using methylene chloride/Hexane to furnish the required product. Yield 0.920 g (81.81%).

R_{f} 0.21 (100% DCM); ¹HNMR (DMSO-d₆): δ 2.26 (s, 3H), 5.08 (s, 2H), 5.22 (s, 2H), 5.25 (s, 2H), 6.19 (s, 1H), 6.47-6.48 (d, 1H), 6.89-6.90 (d, 1H), 7.25-7.47 (m, 17H), 7.78-7.79 (d, 1H), 7.90-7.94 (dd, 1H).

### Compound No. 7: (2-hydroxy-5-(3,5,7-trihydroxy-4-oxo-4H-chromen-2-yl) phenoxy)methyl pivalate

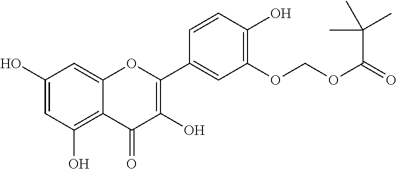

10% Pd/C on charcoal (0.05 g, 10% w/w) was added to a suspension of (2-(Benzyloxy)-5-(3,7-bis(benzyloxy)-5-hydroxy-4-oxo-4H-chromen-2-yl)phenoxy)methyl pivalate (Compound No. 1; 0.5 g, 0.72 mmol) in THF (20 ml) at 25° C. The resulting mixture was shaken under a hydrogen atmosphere (100 psi) for 6 hours. The resultant mixture was filtered over celite 545 to remove Pd/C. The filtrate was evaporated to afford crude product The crude product was purified by column chromatography (60-120 mesh silica gel) using Methylene chloride/Methanol as eluent to furnish the required product. Yield 0.18 g (59.4%).

R_{f} 0.2 (30% MeOH/DCM); ¹H NMR (DMSO-d₆): δ 1.31 (s, 9H), 6.18 (s, 1H), 6.40 (s, 1H, minor isomer), 6.45 (s, 1H, major isomer), 7.04-7.07 (d, 1H, major isomer), 7.17-7.20 (d, 1H, minor isomer), 7.60-7.63 (d, 1H, minor isomer), 7.8 (s, 1H, major isomer), 7.85 (s, 1H, minor isomer), 7.91-7.94 (d, 1H, major isomer), 9.55 (s, 1H, major isomer), 9.71 (s, 1H, minor isomer), 10.02 (s, 1H, minor isomer), 10.43 (s, 1H, major isomer), 10.80 (s, 1H, major isomer), 10.86 (s, 1H, minor isomer); MS (ES−) m/z 415.2 (M−H).

### Compound No. 8: (2-hydroxy-5-(3,5,7-trihydroxy-4-oxo-4H-chromen-2-yl) phenoxy)methyl acetate

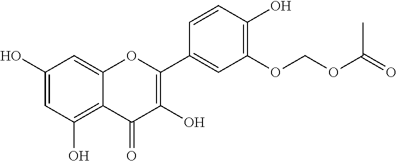

10% Pd/C on charcoal (0.05 g, 20% w/w) was added to a suspension of 2-(Benzyloxy)-5-(3,7-bis(benzyloxy)-5-hydroxy-4-oxo-4H-chromen-2-yl)phenoxy)methyl acetate (Compound No. 2; 0.5 g, 0.77 mmol) in THF:Ethanol (1:1) (20 ml) at 25° C. The resulting mixture was shaken under a hydrogen atmosphere (45 psi) for 5 hours. The resultant mixture was filtered on celit 545 to remove Pd/C. Filtrate was evaporated to afford crude product. The crude product was purified by column chromatography (60-120 mesh silica gel) using Methylene chloride/Methanol as eluent to furnish the required product. Yield 0.220 g (75.86%). (The compound was isolated as THF solvate as seen in ¹ HNMR.

R_{f} 0.3 (30% MeOH/DCM); ¹H NMR (DMSO-d₆): δ 1.74 (q, 4H, THF), 2.27 (s, 3H), 3.58 (q, 4H, THF), 6.18 (s, 1H), 6.44 (d, 1H, both isomer), 7.05-7.08 (d, 1H, major isomer), 7.15-7.18 (d, 1H, minor isomer), 7.58-7.61 (d, 1H, minor isomer), 7.77 (s, 1H, minor isomer), 7.86 (s, 1H, major isomer), 7.92-7.95 (d, 1H, major isomer), 9.58 (s, 1H, major isomer), 9.70 (s, 1H, minor isomer), 10.01 (s, 1H, minor isomer), 10.4 (s, 1H, major isomer), 10.81 (s, 1H, major isomer), 10.86 (s, 1H, minor isomer).

### Compound No. 13: (2-(Benzyloxy)-5-(3,7-bis(benzyloxy)-5-hydroxy-4-oxo-4H-chromen-2-yl) phenoxy)methyl butyrate

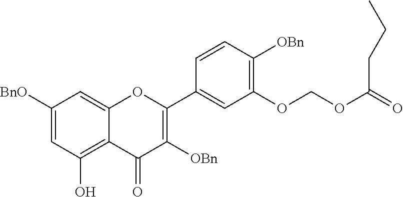

Chloro methylbutyrate (0.28 gm, 2.0 mmol) was added drop wise to a suspension of 3,7-bis(benzyloxy)-2-(4-(benzyloxy)-3-hydroxyphenyl)-5-hydroxy-4H-chromen-4-one [(III, R₁=benzyl) 1 g, 1.7 mmol], triethylamine (0.36 ml, 2.6 mmol) in methylene chloride (20 ml), at 25° C. The resulting mixture was stirred at 25° C. for ten minutes and further for 5 hour at 40-42° C. The reaction mixture was diluted with methylene chloride (25 ml) and water (50 ml). The organic layer was separated, washed with water, dried over anhydrous sodium sulphate and evaporated to give crude product. The crude product was purified by crystallization using methylene chloride/Hexane to furnish the required product.

Yield 0.500 g (42.73%). R_{f} 0.73 (30% EtOAC/Pet.ether); ¹HNMR (DMSO-d₆): δ 0.85-0.90 (t, 3H), 1.57-1.64 (m, 2H), 2.55 (m, 2H), 5.08 (s, 2H), 5.23 (s, 2H), 5.25 (s, 2H), 6.48 (s, 1H), 6.89 (s, 1H), 7.31-7.46 (m, 19H), 7.79 (s, 1H), 7.92-7.95 (d, 1H); HPLC purity=94.9%.

### Compound No. 14: (2-hydroxy-5-(3,5,7-trihydroxy-4-oxo-4H-chromen-2-yl)phenoxy)methyl butyrate

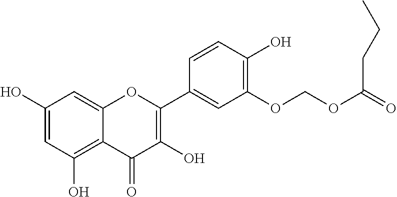

Pd/C 10% on charcoal (0.04 g, 20% w/w) was added to a suspension of 2-(Benzyloxy)-5-(3,7-bis(benzyloxy)-5-hydroxy-4-oxo-4H-chromen-2-yl)phenoxy)methyl butyrate (Compound No. 13; 0.20 g, 0.29 mmol) in THF:Ethanol (1:1) (10 ml) at 25° C. The resulting mixture was shaken under a hydrogen atmosphere (pH₂ 45 psi) for 5 hours. The resultant mixture was filtered on celit 545 to remove Pd/C. Filtrate was evaporated to afford crude product. The crude product was purified by column chromatography (60-120 mesh silica gel) using Methylene chloride/Methanol as eluent to furnish the required product.

Yield 0.07 g (58.82%). R_{f} 0.32 (30% MeOH/DCM); ¹HNMR (DMSO-d₆): δ 0.97-1.01 (t, 3H), 1.17-1.22 (m, 2H), 1.64-1.71 (m, 2H), 2.55-2.61 (m, 2H), 6.20-6.21 (s, 1H, both isomer), 6.43-6.44 (d, 1H, minor isomer), 6.46-6.47 (d, 1H, major isomer), 7.07-7.10 (d, 1H, major isomer), 7.15-7.18 (d, 1H, minor isomer), 7.59 (s, 1H, minor isomer), 7.77 (bs, 1H, minor isomer), 7.84 (bs, 1H, major isomer), 7.92-7.95 (bs, 1H, both isomer), 9.57 (s, 1H, both isomer, OH), 9.90 (s, 1H, minor isomer), 10.9 (s, 1H, both isomer), 12.36 (s, 1H, minor isomer), 12.42 (s, 1H, major isomer); HPLC purity=96.0% (85.83+10.25%).

1-19. (canceled)

20. A compound of formula (I), a pharmaceutically acceptable salt, hydrate, or solvate thereof, 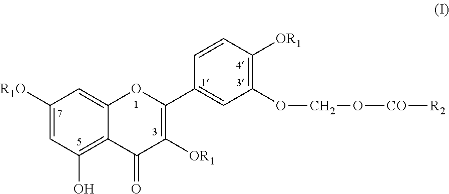
wherein R₁ is selected from hydrogen, benzyl and substituted benzyl; R₂ is selected hydrogen, benzyl, substituted benzyl, linear or branched (C₁-C₆) alkyl, substituted alkyl, aryl, substituted aryl, heterocycle and substituted heterocycle.

21. The compound of formula (I) as claimed in claim 20, wherein the pharmaceutically acceptable salt is selected from ascorbate, acetate, benzoate, citrate, fumarate, gluconate, glutamate, hydrochloride, hydrogen sulfate, lactate, oxalate, phosphate, diphosphate, stearate, succinate, sulfate, tartarate, trifluoroacetate and valerate; Al, Ca, Li, Mg, Na and K salts; halides, amino sugar salt, salt of an amino acid and ammonium.

22. The compound of formula (I) as claimed in claim 20, wherein the ammonium salt is a substituted ammonium salt.

23. The compound of formula (I) as claimed in claim 21, wherein the salt of the amino acid is selected from glycine, alanine, lysine, arginine and guanidine.

24. The compound of formula (I) as claimed in claim 21, wherein the amino sugar salt is selected from N-methyl-D-glucamine, 1-amino-1-deoxy-D-sorbitol, 1-deoxy-1-(methylamino-D-galactilol, 1-deoxy-1-(octylamino)-D-glucitol, 1-deoxy-1-(2-hydroxyethylamino)-D-glucitol, disorbytylamine, D-galactosamine, D-glucosamine, and D-mannosamine.

25. A process for preparation of a compound of formula (I) as claimed in claim 20, 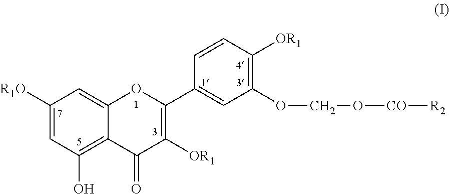
wherein R₁ and R₂ are as defined in claim 20, comprising the steps of:
i) reacting a flavanoid compound of formula (III), 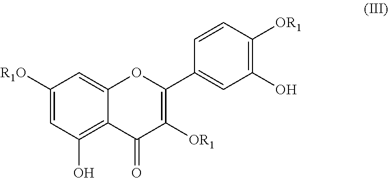
wherein R₁ is selected from benzyl and substituted benzyl with a halo alkyl ester of Formula (V), 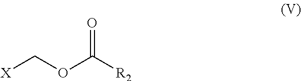
wherein X is selected from chloro, bromo, and iodo and R₂ is selected from hydrogen, benzyl, substituted benzyl, linear or branched (C₁-C₆) alkyl, substituted alkyl, aryl, substituted aryl, heterocycle and substituted heterocycle in presence of a base and an aprotic solvent to obtain a compound of formula (I), wherein R₁ is benzyl or substituted benzyl; and optionally
ii) subjecting the compound of formula (I), as obtained in step (i), wherein R₁ is benzyl or substituted benzyl to catalytic hydrogenation in presence of an organic solvent and a hydrogenation catalyst to obtain a compound of formula (I), wherein R₁ is hydrogen.

26. The process as claimed in claim 25, further comprising the step of converting the compound of formula (I), wherein R₁ is benzyl, substituted benzyl or hydrogen to a pharmaceutically acceptable salt, hydrate or solvate thereof.

27. The process as claimed in claim 26, wherein the pharmaceutically acceptable salt is selected from ascorbate, acetate, benzoate, citrate, fumarate, gluconate, glutamate, hydrochloride, hydrogen sulfate, lactate, oxalate, phosphate, diphosphate, stearate, succinate, sulfate, tartarate, trifluoroacetate and valerate; Al, Ca, Li, Mg, Na and K salts; halide; salt of an amino acid; amino sugar salt and ammonium.

28. The process of formula (I) as claimed in claim 27, wherein the ammonium salt is a substituted ammonium salt.

29. The process of formula (I) as claimed in claim 27, wherein the salt of the amino acid is selected from glycine, alanine, lysine, arginine and guanidine.

30. The process as claimed in claim 27, wherein the amino sugar salt is selected from N-methyl-D-glucamine, 1-amino-1-deoxy-D-sorbitol, 1-deoxy-1-(methylamino-D-galactilol, 1-deoxy-1-(octylamino)-D-glucitol, 1-deoxy-1-(2-hydroxyethylamino)-D-glucitol, disorbytylamine, D-galactosamine, D-glucosamine, and D-mannosamine.

31. The process as claimed in claim 25, wherein aprotic solvent is selected from N,N-dimethylformamide, N,N-dimethylacetamide, dioxane, tetrahydrofuran, acetonitrile, acetone, dichloromethane and dichloroethane.

32. The process as claimed in claim 25, wherein the halo alkyl ester of formula (V) is employed in a proportion of between 1 to 1.5 moles per mole of the flavanoid compound of formula (III).

33. The process as claimed in claim 25, wherein base is an organic or an inorganic base.

34. The process as claimed in claim 33, wherein the organic base is selected from a tertiary amine.

35. The process as claimed in claim 34, wherein the tertiary amine is selected from alkyl amines, pyridine, 2,6-lutidine, N-methyl-morpholine, 4-dimethylaminopyridine, and N,N-dimethylaniline.

36. The process as claimed in claim 33, wherein the inorganic base is selected from alkali metal carbonates.

37. The process as claimed in claim 36, wherein the alkali metal carbonate is selected from alkali metal bicarbonates.

38. The process as claimed in claim 37, wherein alkali metal bicarbonate is selected from sodium bicarbonate and potassium bicarbonate.

39. The process as claimed in claim 25, wherein the base is employed in proportions of between 1.0 to 2.0 moles per mole of the flavanoid compound of formula (III).

40. The process as claimed in claim 25, wherein the reaction of the flavanoid compound of formula (III) and the halo alkyl ester of formula (IV) is carried out at a temperature of from about 10° C. to about 80° C.

41. The process as claimed in claim 25, wherein the organic solvent employed in step (ii) is selected from organic acids, cycloethers, alcohols and mixtures thereof.

42. The process as claimed in claim 25, wherein the organic acid is selected from acetic acid, butenic acid and propionic acid.

43. The process as claimed in claim 41, wherein the cycloether is selected from tetrahydrofuran and dioxanes.

44. The process as claimed in claim 41, wherein the alcohol is selected from methanol, ethanol and propanol.

45. The process as claimed in claim 25, wherein the hydrogenation catalyst is selected from platinum supported on carbon and palladium supported on carbon.

46. The process as claimed in claim 25, wherein the catalytic hydrogenation is carried out under a hydrogen pressure of from 0 to 200 psi.

47. A pharmaceutical composition comprising a therapeutically effective amount of a compound of formula (I) according to claim 20 and at least one pharmaceutically acceptable carrier, adjuvant or diluent.

48. The pharmaceutical composition of claim 47, in the form of a unit dosage form for parenteral or oral administration.

49. A method of treating a mammal with cancer, multi-drug resistant cancer, or viral infection comprising administering to the mammal an effective amount of a compound of formula (I) as claimed in claim 20.
